(19) United States
(12) Patent Application Publication
(10) Pub. No.: US 2014/0187978 A1
(43) Pub. Date: Jul. 3, 2014

(54) Intravascular Devices Having Information Stored Thereon And/Or Wireless Communication Functionality, Including Associated Devices, Systems, And Methods

(71) Applicant: Volcano Corporation, San Diego, CA (US)

(72) Inventors: Bret C. Millett, Folsom, CA (US); Paul Douglas Corl, Palo Alto, CA (US)

(73) Assignee: Volcano Corporation, San Diego, CA (US)

(21) Appl. No.: 14/133,331

(22) Filed: Dec. 18, 2013

Related U.S. Application Data
(60) Provisional application No. 61/747,140, filed on Dec. 28, 2012.

(51) Int. Cl. A61B 5/0215

(52) U.S. Cl. CPC A61B 5/0215
USPC 600/486

(57) ABSTRACT

Intravascular devices, systems, and methods are disclosed. In some embodiments, the intravascular devices are guide-wires that include one or more sensing components and a sensor control module that stores information about the guide-wire. In some instances, the information about the guide-wire stored in the sensor control module is calibration information for a sensing component of the guide-wire. In some embodiments, the intravascular devices are guide-wires that include wireless communication functionality. In some instances, the guide-wires include one or more antennas adjacent a proximal portion of the guide-wire. In some instances, the guide-wires include passive radio frequency devices integrated into the guide-wire. Systems associated with such intravascular devices are disclosed. Methods of using such devices and systems are also disclosed.

### CROSS-REFERENCE TO RELATED APPLICATIONS

The present application claims priority to and the benefit of U.S. Provisional Patent Application No. 61/747,140, filed Dec. 28, 2012, which is hereby incorporated by reference in its entirety.

### TECHNICAL FIELD

The present disclosure relates to intravascular devices, systems, and methods. In some embodiments, the intravascular devices are guide-wires that include one or more sensing components and memory storing information about the guide-wire. In some embodiments, the intravascular devices are guide-wires that include wireless communication functionality.

### BACKGROUND

Heart disease is very serious and often requires emergency operations to save lives. A main cause of heart disease is the accumulation of plaque inside the blood vessels, which eventually occludes the blood vessels. Common treatment options available to open up the occluded vessel include balloon angioplasty, rotational atherectomy, and intravascular stents. Traditionally, surgeons have relied on X-ray fluoroscopic images that are planar images showing the external shape of the silhouette of the lumen of blood vessels to guide treatment. Unfortunately, with X-ray fluoroscopic images, there is a great deal of uncertainty about the exact extent and orientation of the stenosis responsible for the occlusion, making it difficult to find the exact location of the stenosis. In addition, though it is known that restenosis can occur at the same place, it is difficult to check the condition inside the vessels after surgery with X-ray.

A currently accepted technique for assessing the severity of a stenosis in a blood vessel, including ischemia causing lesions, is fractional flow reserve (FFR). FFR is a calculation of the ratio of a distal pressure measurement (taken on the distal side of the stenosis) relative to a proximal pressure measurement (taken on the proximal side of the stenosis). FFR provides an index of stenosis severity that allows determination as to whether the blockage limits blood flow within the vessel to an extent that treatment is required. The normal value of FFR in a healthy vessel is 1.00, while values less than about 0.80 are generally deemed significant and require treatment.

Often intravascular catheters and guide-wires are utilized to measure the pressure within the blood vessel, visualize the inner lumen of the blood vessel, and/or otherwise obtain data related to the blood vessel. To date, guide-wires containing pressure sensors, imaging elements, and/or other electronic, optical, or electro-optical components have suffered from reduced performance characteristics compared to standard guide-wires that do not contain such components. For example, the handling performance of previous guide-wires containing electronic components have been hampered, in some instances, by the need to physically couple the proximal end of the device to a communication line in order to obtain data from the guide-wire, the limited space available for the core wire after accounting for the space needed for the conductors or communication lines of the electronic component(s), the stiffness and size of the rigid housing containing the electronic component(s), and/or other limitations associated with providing the functionality of the electronic components in the limited space available within a guide-wire.

Accordingly, there remains a need for improved intravascular devices, systems, and methods that include one or more electronic, optical, or electro-optical sensing components along with memory for storing information about the guide-wire and/or one or more components that facilitate wireless communication between the guide-wire and another device.

### SUMMARY

Embodiments of the present disclosure are directed to intravascular devices, systems, and methods.

In one embodiment, a guide-wire is provided. The guide-wire comprises: an elongate flexible element having a proximal portion and a distal portion, the elongate flexible element having an outer diameter of 0.018″ or less; a pressure sensing component coupled to the distal portion of the elongate flexible element; a sensor control module coupled to the elongate flexible element, the sensor control module being in electrical communication with the pressure sensing component and storing information about the pressure sensing component; and at least one conductor having a proximal section and a distal section, wherein the distal section of the at least one conductor is coupled to the sensor control module and the proximal section of the at least one conductor is coupled to at least one connector. In some instances, the sensor control module includes an electrically erasable programmable read-only memory (EEPROM). In some implementations, the information about the pressure sensing component includes calibration information. In some embodiments, three to five conductors are utilized and coupled to three to five connectors, each comprising a conductive band.

In another embodiment, an intravascular pressure-sensing system is provided. The system comprises: a pressure-sensing guide-wire having features similar to those described above; a processing system configured to receive the information originating at the sensor; and an interface configured to communicatively couple the sensor to the processing system such that the sensor output is conditioned and communicated to the processing system. In some instances, the interface comprises a communication cable that includes a first connector portion for interfacing with the at least one connector of the pressure-sensing guide-wire and a second connector portion for interfacing with a component of the processing system. In some embodiments, the component of the processing system is a patient interface module (PIM).

In another embodiment a method is provided that includes: obtaining information about a pressure sensing component of a pressure-sensing guide-wire having an outer diameter of 0.018″ or less from a sensor control module coupled to a pressure-sensing guide-wire, the pressure sensing component being coupled to the distal portion of the pressure-sensing guide-wire; and normalizing data received from the pressure sensing component based on the information about the pressure sensing component stored in the memory of the sensor control module. In some instances, the information about the pressure sensing component includes calibration information about the pressure sensing component.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic, schematic side view of an intravascular device according to an embodiment of the present disclosure.

FIG. 2 is diagrammatic cross-sectional side view of an intravascular device according to an embodiment of the present disclosure.

FIG. 3 is a diagrammatic, schematic view of an intravascular system according to an embodiment of the present disclosure.

FIG. 4 is a diagrammatic, schematic view of an intravascular system similar to that of FIG. 3, but illustrating an alternative embodiment of the present disclosure.

FIG. 5 is a diagrammatic, schematic view of an intravascular system similar to those of FIGS. 3 and 4, but illustrating an alternative embodiment of the present disclosure.

FIG. 6 is a diagrammatic, schematic view of a plurality of different mounting options for components of the intravascular devices of the present disclosure.

FIG. 7 is a diagrammatic, schematic view of an intravascular system similar to those of FIGS. 3-5, but illustrating an alternative embodiment of the present disclosure.

FIG. 8 is a diagrammatic, side view of an intravascular device according to another embodiment of the present disclosure.

FIG. 9 is a diagrammatic, top view of a component of the distal portion of the intravascular device of FIG. 8.

FIG. 10 is a diagrammatic, side view a distal portion of the intravascular device shown in FIG. 8 being coupled to a plurality of different intravascular devices in accordance with the present disclosure.

FIG. 11 is a diagrammatic, schematic view of an intravascular device positioned within the body of a patient in communication with a hemostat system according to an embodiment of the present disclosure.

FIG. 12 is a flow chart illustrating a method of performing an intravascular procedure according to an embodiment of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

As used herein, “flexible elongate member” or “elongate flexible member” includes at least any thin, long, flexible structure that can be inserted into the vasculature of a patient. While the illustrated embodiments of the “flexible elongate members” of the present disclosure have a cylindrical profile with a circular cross-sectional profile that defines an outer diameter of the flexible elongate member, in other instances all or a portion of the flexible elongate members may have other geometric cross-sectional profiles (e.g., oval, rectangular, square, elliptical, etc.) or non-geometric cross-sectional profiles. Flexible elongate members include, for example, guide-wires and catheters. In that regard, catheters may or may not include a lumen extending along its length for receiving and/or guiding other instruments. If the catheter includes a lumen, the lumen may be centered or offset with respect to the cross-sectional profile of the device.

In most embodiments, the flexible elongate members of the present disclosure include one or more electronic, optical, or electro-optical components. For example, without limitation, a flexible elongate member may include one or more of the following types of components: a pressure sensor, a temperature sensor, an imaging element, an optical fiber, an ultrasound transducer, a reflector, a minor, a prism, an ablation element, an RF electrode, a conductor, and/or combinations thereof. Generally, these components are configured to obtain data related to a vessel or other portion of the anatomy in which the flexible elongate member is disposed. Often the components are also configured to communicate the data to an external device for processing and/or display. In some aspects, embodiments of the present disclosure include imaging devices for imaging within the lumen of a vessel, including both medical and non-medical applications. However, some embodiments of the present disclosure are particularly suited for use in the context of human vasculature. Imaging of the intravascular space, particularly the interior walls of human vasculature can be accomplished by a number of different techniques, including ultrasound (often referred to as intravascular ultrasound (“IVUS”) and intracardiac echocardiography (“ICE”)) and optical coherence tomography (“OCT”). In other instances, infrared, thermal, or other imaging modalities are utilized.

The electronic, optical, and/or electro-optical components of the present disclosure are often disposed within a distal portion of the flexible elongate member. As used herein, “distal portion” of the flexible elongate member includes any portion of the flexible elongate member from the mid-point to the distal tip. As flexible elongate members can be solid, some embodiments of the present disclosure will include a housing portion at the distal portion for receiving the electronic components. Such housing portions can be tubular structures attached to the distal portion of the elongate member. Some flexible elongate members are tubular and have one or more lumens in which the electronic components can be positioned within the distal portion.

The electronic, optical, and/or electro-optical components and the associated communication lines are sized and shaped to allow for the diameter of the flexible elongate member to be very small. For example, the outside diameter of the elongate member, such as a guide-wire or catheter, containing one or more electronic, optical, and/or electro-optical components as described herein are between about 0.0007″ (0.0178 mm) and about 0.118″ (3.0 mm), with some particular embodiments having outer diameters of approximately 0.014″ (0.3556 mm) and approximately 0.018″ (0.4572 mm)). As such, the flexible elongate members incorporating the electronic, optical, and/or electro-optical component(s) of the present application are suitable for use in a wide variety of lumens within a human patient besides those that are part or immediately surround the heart, including veins and arteries of the extremities, renal arteries, blood vessels in and around the brain, and other lumens.

“Connected” and variations thereof as used herein includes direct connections, such as being glued or otherwise fastened directly to, on, within, etc. another element, as well as indirect connections where one or more elements are disposed between the connected elements.

“Secured” and variations thereof as used herein includes methods by which an element is directly secured to another element, such as being glued or otherwise fastened directly to, on, within, etc. another element, as well as indirect techniques of securing two elements together where one or more elements are disposed between the secured elements.

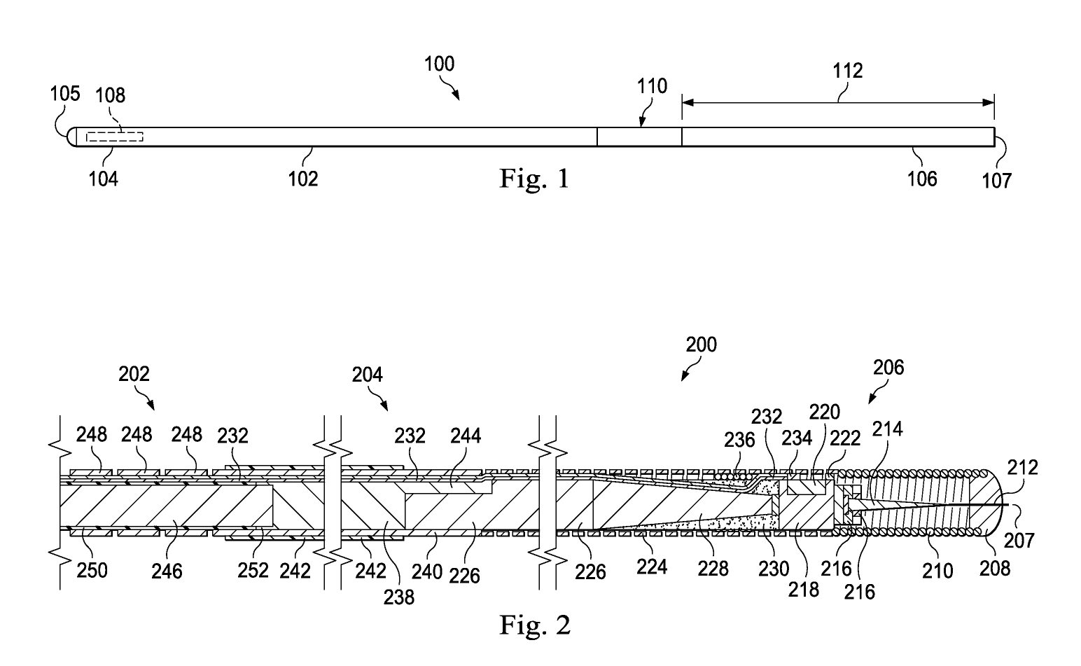

Referring now to FIG. 1, shown therein is a portion of an intravascular device 100 according to an embodiment of the present disclosure. In that regard, the intravascular device 100 includes a flexible elongate member 102 having a distal portion 104 adjacent a distal end 105 and a proximal portion 106 adjacent a proximal end 107. A component 108 is positioned within the distal portion 104 of the flexible elongate member 102 proximal of the distal tip 105. Generally, the component 108 is representative of one or more electronic, optical, or electro-optical components. In that regard, the component 108 is a pressure sensor, a temperature sensor, an imaging element, an optical fiber, an ultrasound transducer, a reflector, a minor, a prism, an ablation element, an RF electrode, a conductor, and/or combinations thereof. The specific type of component or combination of components can be selected based on an intended use of the intravascular device. In some instances, the component 108 is positioned less than 10 cm, less than 5, or less than 3 cm from the distal tip 105. In some instances, the component 108 is positioned within a housing of the flexible elongate member 102. In that regard, the housing is a separate component secured to the flexible elongate member 102 in some instances. In other instances, the housing is integrally formed as a part of the flexible elongate member 102.

The intravascular device 100 also includes a connector 110 adjacent the proximal portion 106 of the device. In that regard, the connector 110 is spaced from the proximal end 107 of the flexible elongate member 102 by a distance 112. Generally, the distance 112 is between 0% and 50% of the total length of the flexible elongate member 102. While the total length of the flexible elongate member can be any length, in some embodiments the total length is between about 1300 mm and about 4000 mm, with some specific embodiments have a length of 1400 mm, 1900 mm, and 3000 mm. Accordingly, in some instances the connector 110 is positioned at the proximal end 107. In other instances, the connector 110 is spaced from the proximal end 107. For example, in some instances the connector 110 is spaced from the proximal end 107 between about 0 mm and about 1400 mm. In some specific embodiments, the connector 110 is spaced from the proximal end by a distance of 0 mm, 300 mm, and 1400 mm.

The connector 110 is configured to facilitate communication between the intravascular device 100 and another device. More specifically, in some embodiments the connector 110 is configured to facilitate communication of data obtained by the component 108 to another device, such as a computing device or processor. Accordingly, in some embodiments the connector 110 is an electrical connector. In such instances, the connector 110 provides an electrical connection to one or more electrical conductors that extend along the length of the flexible elongate member 102 and are electrically coupled to the component 108. In other embodiments, the connector 110 is an optical connector. In such instances, the connector 110 provides an optical connection to one or more optical communication pathways (e.g., fiber optic cable) that extend along the length of the flexible elongate member 102 and are optically coupled to the component 108. Further, in some embodiments the connector 110 provides both electrical and optical connections to both electrical conductor(s) and optical communication pathway(s) coupled to the component 108. In that regard, it should again be noted that component 108 is comprised of a plurality of elements in some instances. In some instances, the connector 110 is configured to provide a physical connection to another device, either directly or indirectly. In other instances, the connector 110 is configured to facilitate wireless communication between the intravascular device 100 and another device. Generally, any current or future developed wireless protocol(s) may be utilized. In yet other instances, the connector 110 facilitates both physical and wireless connection to another device.

As noted above, in some instances the connector 110 provides a connection between the component 108 of the intravascular device 100 and an external device. Accordingly, in some embodiments one or more electrical conductors, one or more optical pathways, and/or combinations thereof extend along the length of the flexible elongate member 102 between the connector 110 and the component 108 to facilitate communication between the connector 110 and the component 108. Generally, any number of electrical conductors, optical pathways, and/or combinations thereof can extend along the length of the flexible elongate member 102 between the connector 110 and the component 108. In some instances, between one and ten electrical conductors and/or optical pathways extend along the length of the flexible elongate member 102 between the connector 110 and the component 108. For the sake of clarity and simplicity, the embodiments of the present disclosure described below include three electrical conductors. However, it is understood that the total number of communication pathways and/or the number of electrical conductors and/or optical pathways is different in other embodiments. More specifically, the number of communication pathways and the number of electrical conductors and optical pathways extending along the length of the flexible elongate member 102 is determined by the desired functionality of the component 108 and the corresponding elements that define component 108 to provide such functionality.

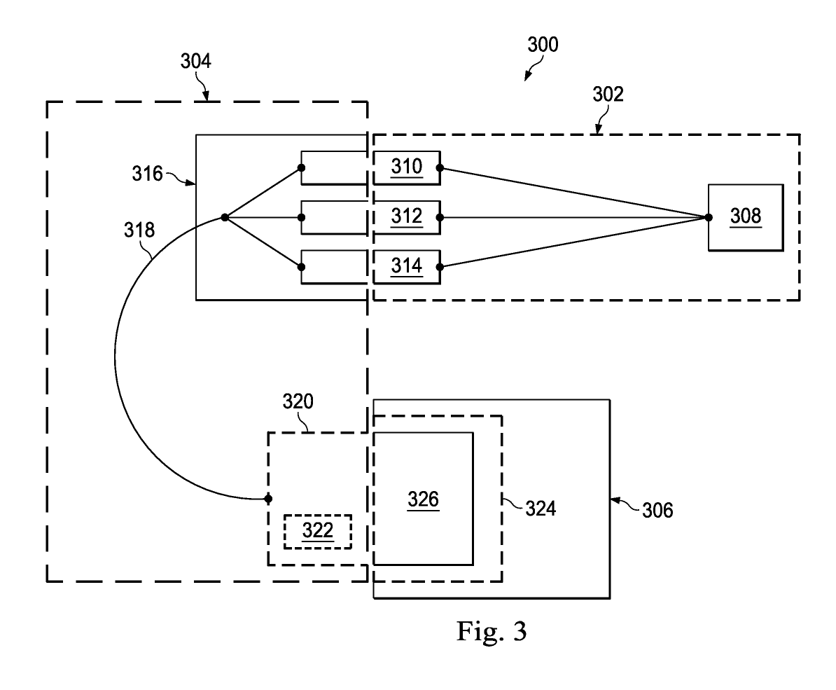

Referring now to FIG. 2, shown therein is a cross-sectional side view of an intravascular device 200 according to an embodiment of the present disclosure. In that regard, the intravascular device 200 is provided as an exemplary embodiment of the type of intravascular device into which the mounting structures, including the associated structural components and methods, described below with respect to FIGS. 3-12 can be implemented. However, it is understood that no limitation is intended thereby and that the concepts of the present disclosure are applicable to a wide variety of intravascular devices, including those described in U.S. Pat. No. 7,967,762 and U.S. Patent Application Publication No. 2009/0088650, each of which is hereby incorporated by reference in its entirety.

As shown in FIG. 2, the intravascular device 200 includes a proximal portion 202, a middle portion 204, and a distal portion 206. Generally, the proximal portion 202 is configured to be positioned outside of a patient, while the distal portion 206 and a majority of the middle portion 204 are configured to be inserted into the patient, including within human vasculature. In that regard, the middle portion 204 and/or distal portion 206 have an outer diameter between about 0.0007″ (0.0178 mm) and about 0.118″ (3.0 mm) in some embodiments, with some particular embodiments having an outer diameter of approximately 0.014″ (0.3556 mm) or approximately 0.018″ (0.4572 mm)). In the illustrated embodiment of FIG. 2, the middle and distal portions 204, 206 of the intravascular device 200 each have an outer diameter of 0.014″ (0.3556 mm).

As shown, the distal portion 206 of the intravascular device 200 has a distal tip 207 defined by an element 208. In the illustrated embodiment, the distal tip 207 has a rounded profile. In some instances, the element 208 is radiopaque such that the distal tip 207 is identifiable under x-ray, fluoroscopy, and/or other imaging modalities when positioned within a patient. In some particular instances, the element 208 is solder secured to a flexible element 210 and/or a flattened tip core 212. In that regard, in some instances the flexible element 210 is a coil spring. The flattened tip core 212 extends distally from a distal portion of a core 214. As shown, the distal core 214 tapers to a narrow profile as it extends distally towards the distal tip 207. In some instances, the distal core 214 is formed of a stainless steel that has been ground down to have the desired tapered profile. In some particular instances, the distal core 214 is formed of high tensile strength 304V stainless steel. In an alternative embodiment, the distal core 214 is formed by wrapping a stainless steel shaping ribbon around a nitinol core. In some embodiments, the distal core 214 is secured to a mounting structure 218 by mechanical interface, solder, adhesive, combinations thereof, and/or other suitable techniques as indicted by reference numerals 216. The mounting structure 218 is configured to receive and securely hold a component 220. In that regard, the component 220 is one or more of an electronic component, an optical component, and/or electro-optical component. For example, without limitation, the component 220 may be one or more of the following types of components: a pressure sensor, a temperature sensor, an imaging element, an optical fiber, an ultrasound transducer, a reflector, a minor, a prism, an ablation element, an RF electrode, a conductor, and/or combinations thereof.

The mounting structure 218 is fixedly secured within the distal portion 206 of the intravascular device 200. As will be discussed below in the context of the exemplary embodiments of FIGS. 3-12, the mounting structure 218 may be fixedly secured to a core wire (i.e., a single core running along the length of the mounting structure), flexible elements or other components surrounding at least a portion of the mounting structure (e.g., coils, polymer tubing, etc.), and/or other structure(s) of the intravascular device positioned adjacent to the mounting structure. In the illustrated embodiment, the mounting structure is disposed at least partially within flexible element 210 and/or a flexible element 224 and secured in place by an adhesive or solder 222. In some embodiments, the mounting structure 218 is disposed entirely within flexible element 210 and/or flexible element 224. In some instances, the flexible elements 210 and 224 are flexible coils. In one particular embodiment, the flexible element 224 is ribbon coil covered with a polymer coating. For example, in one embodiment the flexible element 224 is a stainless steel ribbon wire coil coated with polyethylene terephthalate (PET). In another embodiment, the flexible element is a polyimide tubing that has a ribbon wire coil embedded therein. An adhesive is utilized to secure the mounting structure 218 to the flexible element 210 and/or the flexible element 224 in some implementations. Accordingly, in some instances the adhesive is urethane acrylate, cyanoacrylate, silicone, epoxy, and/or combinations thereof.

The mounting structure 218 is also secured to a core 226 that extends proximally from the mounting structure towards the middle portion 204 of the intravascular device 200. In that regard, core 226 and distal core 214 are integrally formed in some embodiments such that a continuous core passes through the mounting structure. In the illustrated embodiment, a portion 228 of the core 226 tapers as it extends distally towards mounting structure 218. However, in other embodiments the core 226 has a substantially constant profile along its length. In some implementations, the diameter or outer profile (for non-circular cross-sectional profiles) of core 226 and core 214 are the same Like distal core 214, the core 226 is fixedly secured to the mounting structure 218. In some instances, solder and/or adhesive is used to secure the core 226 to the mounting structure 218. In the illustrated embodiment, solder/adhesive 230 surrounds at least a part of the portion 228 of the core 226. In some instances, the solder/adhesive 230 is the solder/adhesive 222 used to secure the mounting structure 218 to the flexible element 210 and/or flexible element 224. In other instances, solder/adhesive 230 is a different type of solder or adhesive than solder/adhesive 222. In one particular embodiment, adhesive or solder 222 is particularly suited to secure the mounting structure 218 to flexible element 210, while solder/adhesive 230 is particularly suited to secure the mounting structure to flexible element 224.

A communication cable 232 extends along the length of the intravascular device 200 from the proximal portion 202 to the distal portion 206. In that regard, the distal end of the communication cable 232 is coupled to the component 220 at junction 234. The type of communication cable utilized is dependent on the type of electronic, optical, and/or electro-optical components that make up the component 220. In that regard, the communication cable 232 may include one or more of an electrical conductor, an optical fiber, and/or combinations thereof. Appropriate connections are utilized at the junction 234 based on the type of communication lines included within communication cable 232. For example, electrical connections are soldered in some instances, while optical connections pass through an optical connector in some instances. In some embodiments, the communication cable 232 is a trifilar structure, a bifilar structure, a single conductor (which may be a conductive core or a conductor separate from the core). Further, it is understood that all and/or portions of each of the proximal, middle, and/or distal portions 202, 204, 206 of the intravascular device 200 may have cross-sectional profiles as shown in FIGS. 2-5 of U.S. Provisional Patent Application No. 61/665,697 filed on Jun. 28, 2012, which is hereby incorporated by reference in its entirety.

Further, in some embodiments, the proximal portion 202 and/or the distal portion 206 incorporate spiral ribbon tubing as disclosed in U.S. Provisional Patent Application No. 61/665,697 filed on Jun. 28, 2012. In some instances, the use of such spiral ribbon tubing allows a further increase in the available lumen space within the device. For example, in some instances use of a spiral ribbon tubing having a wall thickness between about 0.001″ and about 0.002″ facilitates the use of a core wire having an outer diameter of at least 0.0095″ within a 0.014″ outer diameter guide-wire using a trifilar with circular cross-sectional conductor profiles. The size of the core wire can be further increased to at least 0.010″ by using a trifilar with the flattened oblong cross-section conductor profiles. The availability to use a core wire having an increased diameter allows the use of materials having a lower modulus of elasticity than a standard stainless steel core wire (e.g., superelastic materials such as Nitinol or NiTiCo are utilized in some instances) without adversely affecting the handling performance or structural integrity of the guide-wire and, in many instances, provides improvement to the handling performance of the guide-wire, especially when a superelastic material with an increased core diameter (e.g., a core diameter of 0.0075″ or greater) is utilized within the distal portion 206.

The distal portion 206 of the intravascular device 200 also optionally includes at least one imaging marker 236. In that regard, the imaging marker 236 is configured to be identifiable using an external imaging modality, such as x-ray, fluoroscopy, angiograph, CT scan, MRI, or otherwise, when the distal portion 206 of the intravascular device 200 is positioned within a patient. In the illustrated embodiment, the imaging marker 236 is a radiopaque coil positioned around the tapered distal portion 228 of the core 226. Visualization of the imaging marker 236 during a procedure can give the medical personnel an indication of the size of a lesion or region of interest within the patient. To that end, the imaging marker 236 can have a known length (e.g., 0.5 cm or 1.0 cm) and/or be spaced from the element 218 by a known distance (e.g., 3.0 cm) such that visualization of the imaging marker 236 and/or the element 218 along with the anatomical structure allows a user to estimate the size or length of a region of interest of the anatomical structure. It is understood that a plurality of imaging markers 236 are utilized in some instances. In that regard, in some instances the imaging markers 236 are spaced a known distance from one another to further facilitate measuring the size or length of the region of interest.

In some instances, a proximal portion of the core 226 is secured to a core 238 that extends through the middle portion 204 of the intravascular device. In that regard, the transition between the core 226 and the core 238 may occur within the distal portion 206, within the middle portion 204, and/or at the transition between the distal portion 206 and the middle portion 204. For example, in the illustrated embodiment the transition between core 226 and core 238 occurs in the vicinity of a transition between the flexible element 224 and a flexible element 240. The flexible element 240 in the illustrated embodiment is a hypotube. In some particular instances, the flexible element is a stainless steel hypotube. Further, in the illustrated embodiment a portion of the flexible element 240 is covered with a coating 242. In that regard, the coating 242 is a hydrophobic coating in some instances. In some embodiments, the coating 242 is a polytetrafluoroethylene (PTFE) coating.

The proximal portion of core 226 is fixedly secured to the distal portion of core 238. In that regard, any suitable technique for securing the cores 226, 238 to one another may be used. In some embodiments, at least one of the cores 226, 238 includes a plunge grind or other structural modification that is utilized to couple the cores together. In some instances, the cores 226, 238 are soldered together. In some instances, an adhesive is utilized to secure the cores 226, 238 together. In some embodiments, combinations of structural interfaces, soldering, and/or adhesives are utilized to secure the cores 226, 238 together. In other instances, the core 226 is not fixedly secured to core 238. For example, in some instances, the core 226 and the core 246 are fixedly secured to the hypotube 240 and the core 238 is positioned between the cores 226 and 246, which maintains the position of the core 238 between cores 226 and 246. In some implementations, the cores 226, 238, and 246 are integrally formed as a single core.

In some embodiments, the core 238 is formed of a different material than the core 226. For example, in some instances the core 226 is formed of nitinol and the core 238 is formed of stainless steel. In other instances, the core 238 and the core 226 are formed of the same material. In some instances the core 238 has a different profile than the core 226, such as a larger or smaller diameter and/or a non-circular cross-sectional profile. For example, in some instances the core 238 has a D-shaped cross-sectional profile. In that regard, a D-shaped cross-sectional profile has some advantages in the context of an intravascular device 200 that includes one or more electronic, optical, or electro-optical component in that it provides a natural space to run any necessary communication cables while providing increased strength than a full diameter core. In other instances, core 238 and core 226 are made of the same material and/or have the same structure profiles such that the cores 226 and 238 form a continuous, monolithic core.

In some instances, a proximal portion of the core 238 is secured to a core 246 that extends through at least a portion of the proximal portion 202 of the intravascular device 200. In that regard, the transition between the core 238 and the core 246 may occur within the proximal portion 202, within the middle portion 204, and/or at the transition between the proximal portion 202 and the middle portion 204. For example, in the illustrated embodiment the transition between core 238 and core 246 is positioned distal of a plurality of conducting bands 248. In that regard, in some instances the conductive bands 248 are portions of a hypotube. Proximal portions of the communication cable 232 are coupled to the conductive bands 248. In that regard, in some instances each of the conductive bands is associated with a corresponding communication line of the communication cable 232. For example, in embodiments where the communication cable 232 consists of a trifilar, each of the three conductive bands 248 are connected to one of the conductors of the trifilar, for example by soldering each of the conductive bands to the respective conductor. Where the communication cable 232 includes optical communication line(s), the proximal portion 202 of the intravascular device 200 includes an optical connector in addition to or instead of one or more of the conductive bands 248. An insulating layer or sleeve 250 separates the conductive bands 248 from the core 246. In some instances, the insulating layer 250 is formed of polyimide.

As noted above, the proximal portion of core 238 is fixedly secured to the distal portion of core 246. In that regard, any suitable technique for securing the cores 238, 246 to one another may be used. In some embodiments, at least one of the cores includes a structural feature that is utilized to couple the cores together. In the illustrated embodiment, the core 238 includes an extension 252 that extends around a distal portion of the core 246. In some instances, the cores 238, 246 are soldered together. In some instances, an adhesive is utilized to secure the cores 238, 246 together. In some embodiments, combinations of structural interfaces, soldering, and/or adhesives are utilized to secure the cores 238, 246 together. In other instances, the core 226 is not fixedly secured to core 238. For example, in some instances and as noted above, the core 226 and the core 246 are fixedly secured to the hypotube 240 and the core 238 is positioned between the cores 226 and 246, which maintains the position of the core 238 between cores 226 and 246. In some embodiments, the core 246 is formed of a different material than the core 238. For example, in some instances the core 246 is formed of Nitinol and/or NiTiCo (nickel-titanium-cobalt alloy) and the core 238 is formed of stainless steel. In that regard, by utilizing a nitinol core within the conductive bands 248 instead of a stainless steel the likelihood of kinking is greatly reduced because of the increased flexibility of the nitinol core compared to a stainless steel core. In other instances, the core 238 and the core 246 are formed of the same material. In some instances the core 238 has a different profile than the core 246, such as a larger or smaller diameter and/or a non-circular cross-sectional profile. In other instances, core 238 and core 246 are made of the same material and/or have the same structure profiles such that the cores 238 and 246 form a continuous, monolithic core.

Additional embodiments of the present disclosure will now be described in the context of FIGS. 3-12. To this end, various implementations of intravascular devices will be described. It is understood that, for the sake of brevity, some details of these intravascular devices will not be explicitly described with respect to each implementation. Those skilled in the art understand that one or more features, including various combinations thereof, described above in the context of the intravascular devices of FIGS. 1 and 2, including the disclosures in the references incorporated by reference, are utilized for the other embodiments of the present disclosure described below. Thus, unless otherwise stated, any one or more features described above may be included in the intravascular devices described below.

Referring now to FIG. 3, shown therein is an intravascular system 300 according to an embodiment of the present disclosure. As shown, the intravascular system 300 includes an intravascular device 302, an interface 304, and a processing system 306. Generally, the interface 304 facilitates communication between the intravascular device 302 and the processing system 306. In the illustrated embodiment, the intravascular device 302 is a guide-wire having an outer diameter of 0.018″, 0.014″, or less. Further, the intravascular device 302 includes a sensing component 308 coupled to a distal portion of the device that is communicatively coupled to a plurality of connectors 310, 312, 314 at a proximal portion of the device. In some instances, the sensing component 308 is electrically coupled to the connectors 310, 312, 314 and the connectors 310, 312, 314 are themselves electrically conductive elements.

The interface 304 communicatively couples the intravascular device 302 to the processing system 306. To that end, the interface 304 includes a custom connector 316 for interfacing with the connectors 310, 312, 314 of the intravascular device. The interface 304 also includes a cable 318 that extends from the custom connector 316 to a modular connector 320. In that regard, the modular connector 320 is configured to interface with the processing system 306. The modular connector 320 includes, or is in communication with a memory unit 322 that stores information about the intravascular device 302 and, in particular, the sensing component 308. In some instances, the memory unit 322 stores device-specific information such as: device ID, usage limit, sensor ID, temperature coefficient, zero offset, scale factor, sensitivity, manufacture date, manufacture time, and manufacture location. In addition, the memory unit 322 may store information related to one or more specific periods of device activation or use, such as: count, date, time, location, system ID, pressure minimum, pressure maximum, velocity minimum, velocity maximum, temperature minimum, temperature maximum, and centered (y/n). To that end, the memory unit 322 can be any suitable type of memory device, including without limitation EEPROM or Flash, stand-alone or embedded, and/or combinations thereof.

The processing system 306 is coupled to the modular connector 320 of the interface 304. In the illustrated embodiment, the processing system 306 includes a patient interface module (PIM) 324 that includes a socket 326 for mating engagement with the modular connector 320. In some implementations, the PIM 324 includes a separate housing from other portions of the processing system 306 that the PIM communicates with, such as a workstation, console, desktop computer, laptop computer, tablet, and/or other processing component. In some implementations, the PIM is integrated into the same housing as one or more other portions of the processing system 306.

While the arrangement of the intravascular system 300 of FIG. 3 is adequate for its intended purpose, it is less than ideal because it requires that the memory unit 322 of the interface 304 be packaged and utilized exclusively with the paired intravascular device 302. For example, in some instances the memory unit 322 carries sensor specific calibration information that is utilized to normalize the output of the paired sensor to behave similarly to every other sensor/guide-wire. Accordingly, it is imperative that the matching interface 304 and intravascular device 302 be used together.

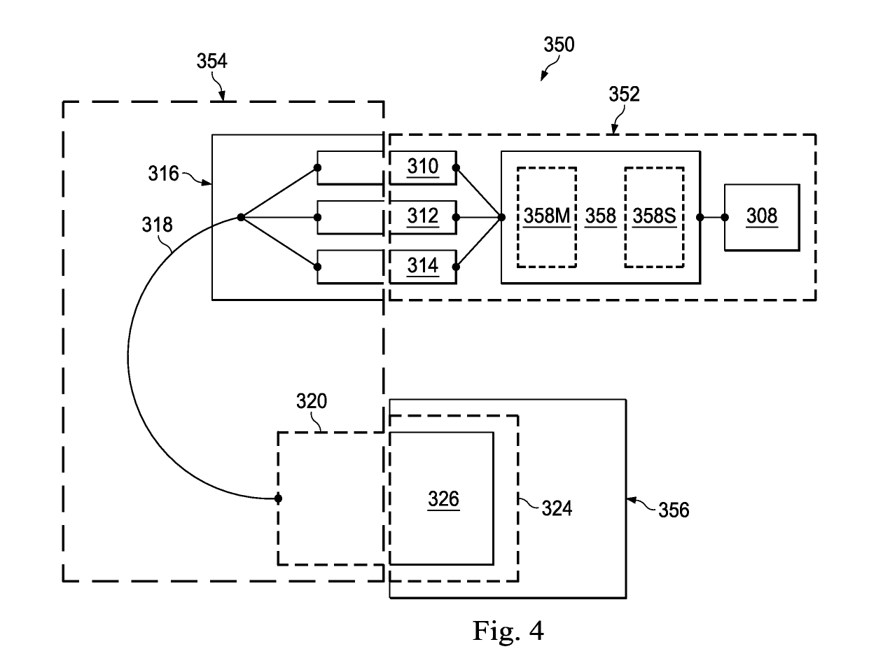

Referring now to FIG. 4, shown therein is an intravascular system 350 according to an embodiment of the present disclosure. As shown, the intravascular system 350 includes an intravascular device 352, a cable/connector interface 354, and a processing system 356. Generally, the cable/connector interface 354 facilitates communication between the intravascular device 352 and the processing system 356. In the illustrated embodiment, the intravascular device 352 is a guide-wire having an outer diameter of 0.018″, 0.014″, or less. Further, the intravascular device 352 includes a sensing component 308 coupled to a distal portion of the device. The intravascular device 352 also includes a memory/normalization module 358. In some implementations, the memory/normalization module 358 is communicatively coupled to the sensing component 308. In other implementations, the memory/normalization module 358 is communicatively isolated from the sensing component 308. The memory/normalization module 358 stores information about the intravascular device 302 and, in particular, the sensing component 308. In some instances, the memory/normalization module 358 stores device-specific information such as: device ID, usage limit, sensor ID, temperature coefficient, zero offset, scale factor, sensitivity, manufacture date, manufacture time, and manufacture location. In addition, the memory/normalization module 358 may store information related to one or more specific periods of device activation or use, such as: count, date, time, location, system ID, pressure minimum, pressure maximum, velocity minimum, velocity maximum, temperature minimum, temperature maximum, and centered (y/n).

In order to be disposed within the intravascular device 352 without adversely affecting performance or usefulness of the intravascular device 352, the memory/normalization module 358 must have a profile that allows it to be positioned within the intravascular device 352 without increasing the outer profile of the intravascular device 352. For example, in instances where the intravascular device 352 has an outer diameter of 0.014″, the memory/normalization module 358 has a height between about 0.02 mm and about 0.075 mm, a width between about 0.125 mm and about 0.35 mm, and a length between about 0.200 mm and about 7.0 mm, however sizes outside these ranges are contemplated. To that end, applicants have found that the memory unit 358 can be any suitable type of memory device, including without limitation EEPROM or Flash, stand-alone or embedded.

To facilitate retrieval of the information from the memory/normalization module 358, the memory/normalization module 358 is communicatively coupled to a plurality of connectors 310, 312, 314 at a proximal portion of the device. In some instances, the sensing component 308 is communicatively coupled to the connectors 310, 312, 314 via memory/normalization module 358. In some instances, the sensing component 308 is electrically coupled to the connectors 310, 312, 314 through memory/normalization module 358 and the connectors 310, 312, 314 are electrically conductive elements. The cable/connector interface 354 communicatively couples the intravascular device 352 to the processing system 356. To that end, the cable/connector interface 354 includes a custom connector 316 for interfacing with the connectors 310, 312, and 314 of the intravascular device. The cable/connector interface 354 also includes a cable 318 that extends from the custom connector 316 to a modular connector 320. In that regard, the modular connector 320 is configured to interface with the processing system 356. In contrast to the embodiment of FIG. 3, the cable/connector interface 354 and, in particular, the modular connector 320 does not include a memory unit, and is device-neutral. Instead, relevant information about the intravascular device 352 is stored in the memory/normalization module 358 integrated into the intravascular device 352 itself. As a result, there is no need for cable/connector interface 354 to be associated with a particular intravascular device 352. Instead, the same cable/connector interface 354 is suitable for use with a plurality of different intravascular devices 352, including devices that may have different calibration and/or operating parameters.

The processing system 356 is coupled to the modular connector 320 of the cable/connector interface 354. In the illustrated embodiment, the processing system 356 includes a patient interface module (PIM) 324 that includes a socket 326 for mating engagement with the modular connector 320. In some implementations, the PIM 324 includes a separate housing from other portions of the processing system 356 that the PIM 324 communicates with, such as a workstation, console, desktop computer, laptop computer, tablet, and/or other processing component. In some implementations, the PIM 324 is integrated into the same housing as one or more other portions of the processing system 356. In use, the processing system 356 is able to obtain any necessary information about the intravascular device 352, including information about the sensing component 308, from the memory/normalization module 358 via the connections provided by the cable/connector interface 354. Accordingly, in instances where the memory/normalization module 358 carries calibration information about the sensing component 308 of the intravascular device 352, the processing system 356 is able to obtain and utilize the calibration information from the memory/normalization module 358 to render accurate measurements based on the data provided by the intravascular device 352.

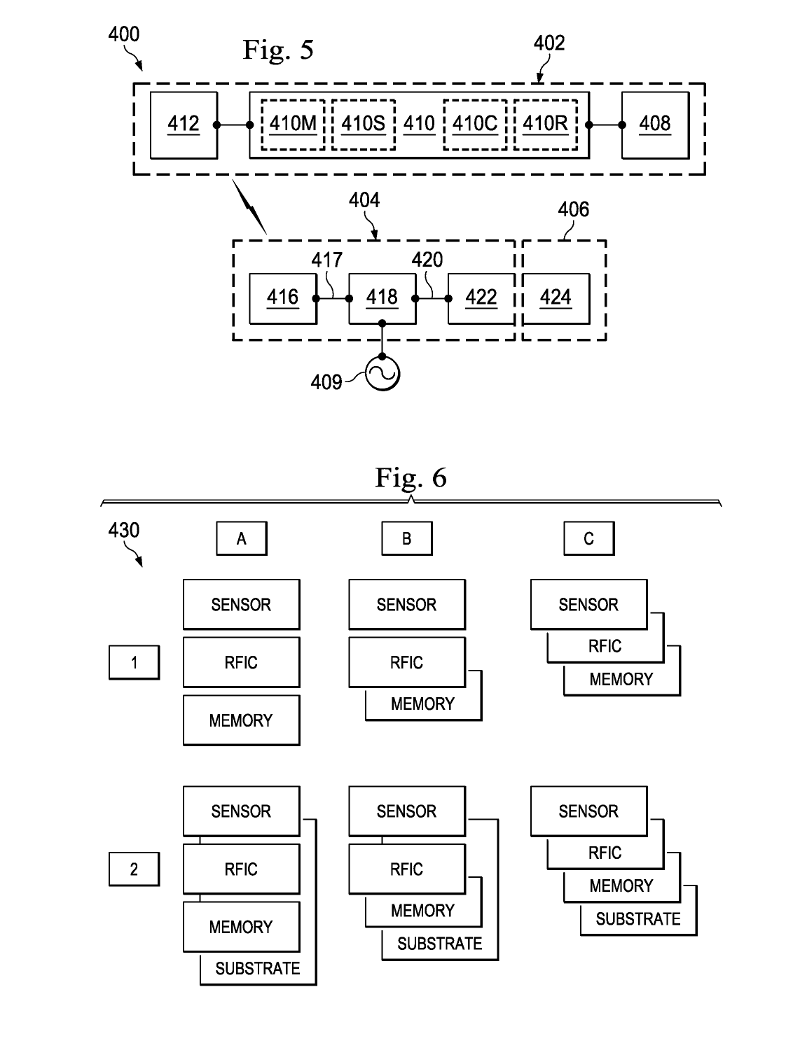

Referring now to FIG. 5, shown therein is an intravascular system 400 according to an embodiment of the present disclosure. As shown, the intravascular system 400 includes an intravascular device 402, a reader interface 404, and a processing system 406. Generally, the reader interface 404 facilitates communication between the intravascular device 402 and the processing system 406. In the illustrated embodiment, the intravascular device 402 is a guide-wire having an outer diameter of 0.018″, 0.014″, or less. Further, the intravascular device 402 includes a sensing component 408 coupled to a distal portion of the device. The intravascular device 402 may also include integrated circuits for data storage, signal conditioning, rectification, energy storage, and telemetry. In some implementations, energy is stored in one or more discrete components. In some implementations, the signal conditioning, communications, and rectification elements are integrated into an application specific integrated circuit.

Generally, the energy storage element(s), the application specific integrated circuit, the memory chip, and the sensor may be arranged in any suitable manner meeting the stringent size requirements for positioning within the distal portion of a guide-wire having an outer diameter of 0.018″, 0.014″, or less. In some implementations, the energy storage element(s), the application specific integrated circuit, the memory chip, and the sensor are positioned one atop another in a vertical stack. In other implementations, the energy storage element(s), the application specific integrated circuit, the memory chip, and the sensor are independently positioned on a substrate. In some implementations, the substrate is fabricated flat and mounted flat within the elongate member. In another implementation the substrate is fabricated flat, and is then wrapped around the core wire. In another implementation, the substrate is fabricated directly on the non-planar surface of the core wire.

A visual representation 430 of exemplary combinations of arrangements for a sensor, RFIC, and memory chip are shown in FIG. 6. In particular, the visual representation 430 includes three columns labeled “A”, “B”, and “C” and two rows labeled “1” and “2”. The “A” column corresponds to arrangements where the sensor, RFIC, and memory chip are mounted separately; the “B” column corresponds to arrangements where the RFIC and memory chip are stacked and mounted separately from the sensor; and the “C” column corresponds to arrangements where the sensor, RFIC, and memory chip are stacked together. The “1” row corresponds to mounting arrangements that do not include a common substrate, while the “2” row represents mounting arrangements where the sensor, RFIC, and memory chip are mounted to a common substrate (e.g., flex circuit, semiconductor substrate, PCB, or otherwise). Accordingly, the sensor, RFIC, and memory chip may be mounted using any of arrangements represented by A-1, A-2, B-1, B-2, C-1, and C-2. It is understood that these arrangements are representative of the different types of stacked, partially stacked, and separated mounting arrangements on a substrate or not that may be implemented in the context of the present disclosure. These concepts can be expanded to any number of components and corresponding combinations of stacked, partially, and separated mounting arrangements on a common substrate, a partially-common substrate (i.e., more than one but less than all components mounted to the same substrate), and no common substrate. Any quantity, combination, and physical arrangement of application specific integrated circuit(s), memory die, discrete component(s), substrate(s), antenna(s), and sensor(s), is collectively referred to herein as the sensor control module 410 and may be located within the intravascular device 402.

The sensor control module 410 stores information about the intravascular device 402 and, in particular, the unique characteristics of the sensing component 408 to which it is paired. In some instances, the sensor control module 410 stores device-specific information such as: device ID, usage limit, sensor ID, temperature coefficient, zero offset, scale factor, sensitivity, manufacture date, manufacture time, and manufacture location. In addition, the sensor control module 410 may store information related to one or more specific periods of device activation or use, such as: count, date, time, location, system ID, pressure minimum, pressure maximum, velocity minimum, velocity maximum, temperature minimum, temperature maximum, centered (y/n), and reader ID.

In order to be disposed within the intravascular device 402 without adversely affecting performance or usefulness of the intravascular device 402, the sensor control module 410 must have a profile that allows it to be positioned within the intravascular device 402 without increasing the outer profile of the intravascular device 402. For example, in instances where the intravascular device 402 has an outer diameter of 0.014″, the sensor control module 410 has a height between about 0.02 mm and about 0.075 mm, a width between about 0.125 mm and about 0.35 mm, and a length between about 0.200 mm and about 7.0 mm, however sizes outside these ranges are contemplated. Further, as described below, the sensor control module 410 of the present embodiment is also suitable for wireless communication with reader interface 404 via a guide-wire antenna 412. To that end, applicants have found that suitable sensor control module 410 can include any suitable type of memory device, including without limitation EEPROM or Flash, stand-alone or embedded, and/or combinations thereof.

To facilitate retrieval of the information from the sensing component 408, the sensor control module 410 is communicatively coupled to at least one guide-wire antenna 412. In some configurations, the guide-wire antenna 412 constitutes the proximal portion of the intravascular device 402. In some embodiments, the guide-wire antenna 412 may be configured to reside completely outside of the patient during a procedure. In another embodiment, the guide-wire antenna 412 might extend from the proximal tip to a distal extent that resides within the patient during a procedure. In some embodiments, the guide-wire antenna 412 is confined exclusively to the middle region, or exclusively to the distal region of the elongate member. The guide-wire antenna 412 may be a monopole, dipole, meandering, straight, helical, or other suitable arrangement.

The reader interface 404 communicatively couples the intravascular device 402 to the processing system 406. In some configurations, the reader interface 404 includes a reader antenna 416 for communicating with the guide-wire antenna 412 of the intravascular device 402. The reader antenna 416 may be positioned or installed in any suitable location in the room within range of the use-envelope of the guide-wire antenna 412. The reader antenna 416 communicates with the reader module 418 via a cable interface. The reader module 418 receives power from a power source 658, which may be an AC outlet, power-over-Ethernet (POE), or suitable power supply. In some instances, the reader module 418 communicates with the processing system 406 through a cable 420 and connector pair. In the illustrated embodiment, the connector 422 is a USB connector that connects to a USB connector of the processing system 406. However, it is understood that essentially any type of wired connection between the reader interface 404 and the processing system 406 may be utilized. In the illustrated embodiment, at least a portion of the reader interface 404 is a patient interface module (PIM). In some implementations, the PIM includes a separate housing from the processing system 406 that the PIM communicates with, such as a workstation, console, desktop computer, laptop computer, tablet, and/or other processing component. In some implementations, the PIM is integrated into the same housing as one or more components of the processing system 406.

Similar to the embodiment of FIG. 4, the reader interface 404 is device-neutral. The relevant information about the intravascular device 402 is stored in the sensor control module 410 and integrated into the intravascular device 402 itself. As a result, there is no need for the reader interface 404 to be associated with any specific intravascular device 402. Instead, the same reader interface 404 is suitable for use with a plurality of intravascular devices 402, including devices that may have different calibration and/or operating parameters. In use, the processing system 406 is able to obtain any necessary information about the intravascular device 402 as described previously.

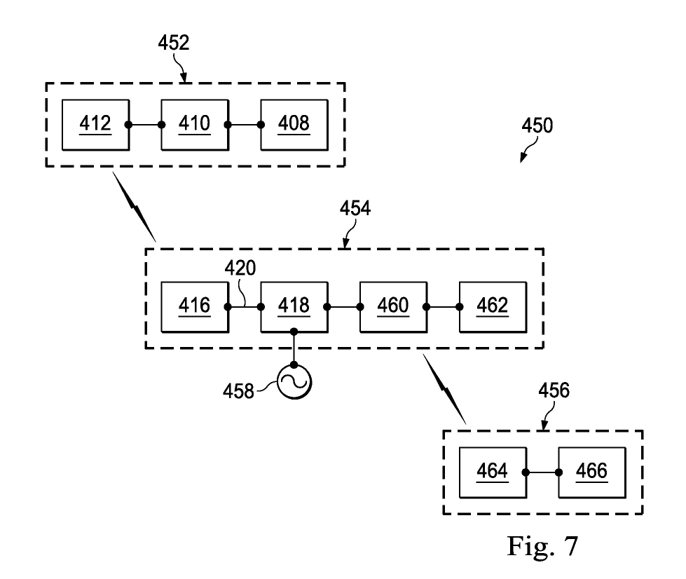

Referring now to FIG. 7, shown therein is an intravascular system 450 according to an embodiment of the present disclosure. As shown, the intravascular system 450 includes an intravascular device 452, a reader interface 454, and a processing system 456. Generally, the reader interface 454 facilitates communication between the intravascular device 452 and the processing system 456. In the illustrated embodiment, the intravascular device 452 is a guide-wire having an outer diameter of 0.018″, 0.014″, or less. Further, the intravascular device 452 includes a sensing component 408 coupled to a distal portion of the device. The intravascular device 452 may also include integrated circuits for data storage, signal conditioning, rectification, energy storage, and telemetry. In some implementations, energy is stored in one or more discrete components. In some implementations, the signal conditioning, communications, and rectification elements are integrated into an application specific integrated circuit and coupled with memory to form a sensor control module 410. In some implementations, the energy storage element(s), the application specific integrated circuit, the memory chip, and the sensor are positioned one atop another in a vertical stack (e.g., arrangement C-1 of FIG. 6). In some implementations, the energy storage element(s), the application specific integrated circuit, the memory chip, and the sensor are independently positioned on a substrate (e.g., arrangement A-2 of FIG. 6). In some implementations, the substrate is fabricated flat and mounted flat within the elongate member. In another implementation the substrate is fabricated flat, and is then wrapped around the core wire. In another implementation, the substrate is fabricated directly on the non-planar surface of the core wire. Any quantity, combination, and physical arrangement of application specific integrated circuit(s), memory die, discrete component(s), substrate(s), antenna(s), and sensor(s) may be located within the elongate unit. The sensor control module 410 stores information about the intravascular device 452 and, in particular, the unique characteristics of the sensing component 408 to which it is paired. In some instances, the sensor control module 410 stores device-specific information such as: device ID, usage limit, sensor ID, temperature coefficient, zero offset, scale factor, sensitivity, manufacture date, manufacture time, and manufacture location. In addition, the sensor control module 410 may store information related to one or more specific periods of device activation or use, such as: count, date, time, location, system ID, pressure minimum, pressure maximum, velocity minimum, velocity maximum, temperature minimum, temperature maximum, centered (y/n), and reader ID.

In order to be disposed within the intravascular device 452 without adversely affecting performance or usefulness of the intravascular device 452, the sensor control module 410 must have a profile that allows it to be positioned within the intravascular device 452 without increasing the outer profile. For example, in instances where the intravascular device 452 has an outer diameter of 0.014″, the sensor control module 410 has a height between about 0.02 mm and about 0.075 mm, a width between about 0.125 mm and about 0.35 mm, and a length between about 0.200 mm and about 7.0 mm, however sizes outside these ranges are contemplated. Further, as described below, the sensor control module 410 of the present embodiment is also suitable for wireless communication with reader interface 454 via a guide-wire antenna 412. To that end, applicants have found that suitable sensor control module 410 can include any suitable type of memory device, including without limitation EEPROM or Flash, stand-alone or embedded.

To facilitate retrieval of the information from the sensing component 408, the sensor control module 410 is communicatively coupled to at least one guide-wire antenna 412. In some configurations, the guide-wire antenna 412 constitutes the proximal portion of the intravascular device 402. In some embodiments, the guide-wire antenna 412 may be configured to reside completely outside of the patient during a procedure. In another embodiment, the guide-wire antenna 412 might extend from the proximal tip to a distal extent that resides within the patient during a procedure. In some embodiments, the guide-wire antenna 412 is confined exclusively to the middle region, or exclusively to the distal region of the elongate member. The guide-wire antenna 412 may be a monopole, dipole, meandering, straight, helical, or other suitable arrangement.

The reader interface 454 communicatively couples the intravascular device 452 to the processing system 456. To that end, the reader interface 454 includes a reader antenna 416 for communicating with the guide-wire antenna 412 of the intravascular device 452; and a link antenna 462 for communicating with the system antenna 464 of the processing system 456 that is in communication with processing component 466. The reader antenna 416 may be positioned or installed in any suitable location in the room within range of the use-envelope of the guide-wire antenna 412. The link antenna 462 may be positioned or installed in any suitable location in the room within range of the system antenna 464, and the same is true for the system antenna 464 relative to the link antenna 462. The reader module 418 may be powered by an AC outlet, by power-over-Ethernet (POE), or by other suitable power supply. The reader module 418 communicates with the telemetry module 460 which communicates with the processing system 456 through the link antenna 462 to system antenna 464 coupling. The communication protocol between the reader interface 454 and the processing system 456 (and between the reader interface 454 and the intravascular device 452) may be one or any combination of industry standard and/or proprietary types (Bluetooth, Wi-Fi, UHF, HF, etc.). In the illustrated embodiment, at least a portion of the reader interface 454 is a patient interface module (PIM). In some implementations, the PIM includes a separate housing from the processing system 456 that the PIM communicates with, such as a workstation, console, desktop computer, laptop computer, tablet, and/or other processing component. In some implementations, the PIM is integrated into the same housing as one or more components of the processing system 456.

Similar to the embodiments of FIGS. 4 and 5, the reader interface 454 is device-neutral. The relevant information about the intravascular device 452 is stored in the sensor control module 410 and integrated into the intravascular device 452 itself. As a result, there is no need for reader interface 454 to be associated with a particular intravascular device 452. Instead, the same reader interface 454 is suitable for use with a plurality of different intravascular devices 452, including devices that may have different calibration and/or operating parameters. In use, the processing system 456 is able to wirelessly obtain any necessary information about the intravascular device 452, including information about the sensing component 408, from the sensor control module 410 via the wireless connection between the reader interface 454 and the intravascular device 452 and the wireless connection between the reader interface 454 and the processing system 456. Accordingly, in instances where the sensor control module 410 carries calibration information about the sensing component 408 of the intravascular device 452, the processing system 456 is able to obtain and utilize the calibration information from the sensor control module 410 to render accurate measurements based on the data provided by the intravascular device 452.

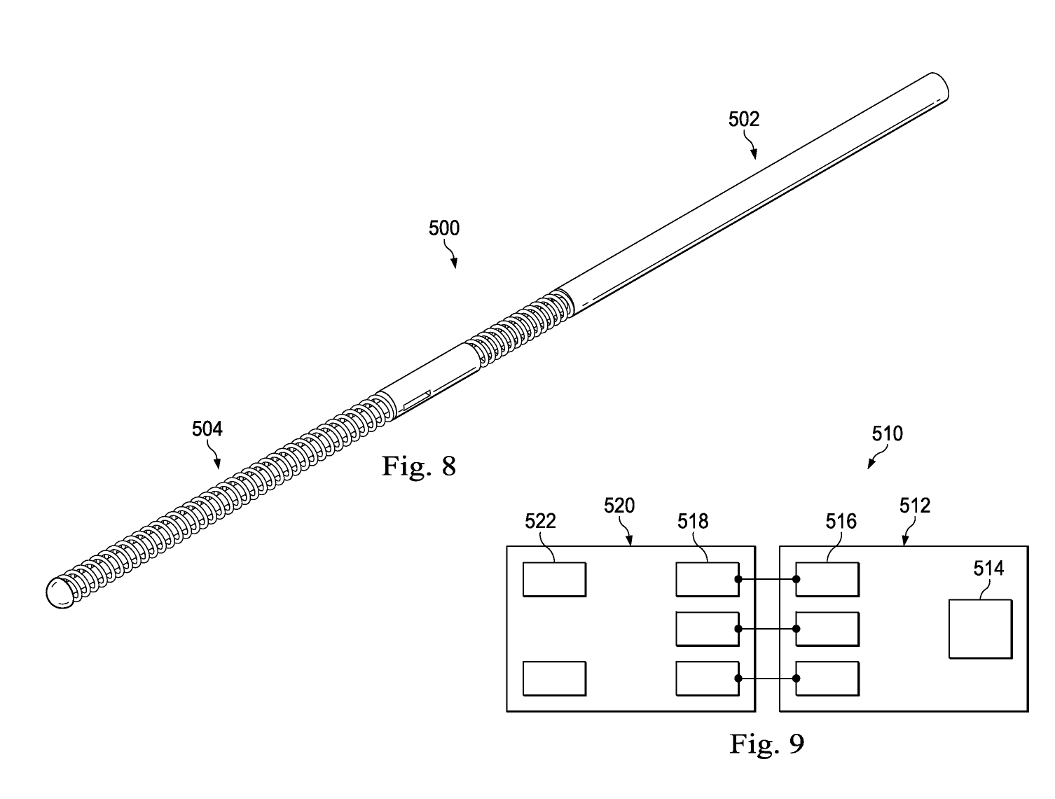

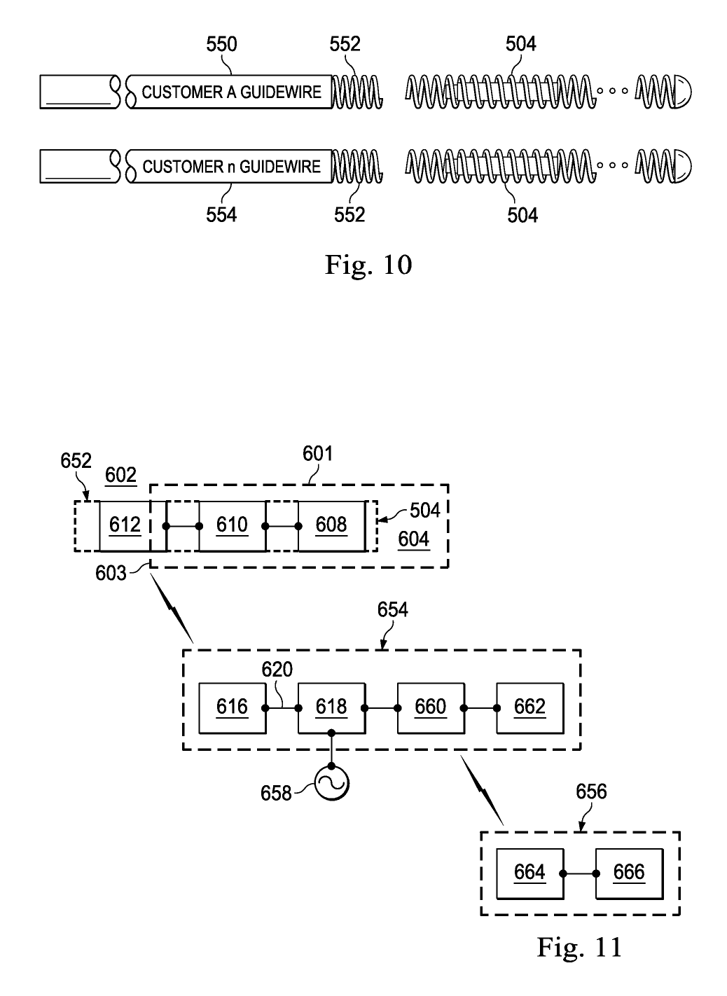

Referring now to FIGS. 8-12, aspects of wireless intravascular devices and associated systems and methods will now be described. Referring more specifically to FIGS. 8 and 9, shown therein are aspects of an intravascular device 500 according to another embodiment of the present disclosure. The intravascular device 500 includes an elongate, flexible proximal portion 502 coupled to an elongate, flexible distal portion 504. In that regard, proximal portion 502 may include one or more features similar to those of the proximal portion 106, and/or proximal portion 202, and/or middle portion 204 described above. Likewise, the distal portion 504 may include one or more features similar to those of distal portion 104 and/or distal portion 206 described above. The distal portion 504 is fixedly secured to the proximal portion 502. To that end, the distal portion 504 may be mechanically coupled, chemically bonded, and/or otherwise secured to the proximal portion 502. For example, in some instances, the distal portion 504 includes a coil structure that is threaded onto a mating coil structure of the proximal portion 502. In addition to or in lieu of the coil structure(s), the distal portion 504 may be welded, and/or bonded to the proximal portion using an adhesive (e.g., epoxy, glue, etc.), solder, and/or other suitable bonding agent. Generally, the distal portion 504 may be coupled to the proximal portion 502 in any suitable way. Further, as discussed below in the context of FIG. 10, in some implementations the distal portion 504 and the proximal portion 502 have a standardized connection arrangement such that various combinations of available distal portions and proximal portions can be easily and conveniently put together to form intravascular devices having features specifically selected based on user preferences, procedure needs, and/or combinations thereof.

In some implementations, the proximal portion 502 is configured to facilitate operation of the intravascular device 500 such that power and data are wirelessly transferred from a reader interface 454 to the proximal portion 502, and data is wirelessly transferred from the proximal portion 502 to the reader interface 454. More specifically, the proximal portion 502 is configured as an antenna to harvest energy and to facilitate operation of electrical, optical, and/or electro-optical components coupled to the proximal portion 502 such that data obtained by the electrical, optical, and/or electro-optical components can be wirelessly communicated to a reader interface 454 during a procedure (i.e., while the distal portion 504 is positioned within the body of a patient) and/or following a procedure (i.e., after the distal portion 504 has been removed from the body of the patient). As described below, in some implementations the proximal portion 502 is configured to receive power from the reader interface 454 and to selectively distribute said energy to the electrical, optical, and/or electro-optical component(s). Due to the wireless connection, there is no need for a connector/cable interface between the proximal portion 502 and a Patient Interface Module (PIM). As a result, the intravascular device is more easily handled and steered by users.

In some implementations, the distal portion 504 is configured to facilitate operation of the intravascular device 500 such that power and data are wirelessly transferred from a reader interface 454 to the distal portion 504, and data is wirelessly transferred from the distal portion 504 to the reader interface 454. More specifically, the distal portion 504 is configured with an antenna to harvest energy and to facilitate operation of electrical, optical, and/or electro-optical components coupled to the distal portion 504 such that data obtained by the electrical, optical, and/or electro-optical components can be wirelessly communicated to a reader interface 454 during a procedure (i.e., while the distal portion 504 is positioned within the body of a patient) and/or following a procedure (i.e., after the distal portion 504 has been removed from the body of the patient). As described below, in some implementations the distal portion 504 is configured to receive power from the reader interface 454 and to selectively distribute said energy to the electrical, optical, and/or electro-optical component(s). Due to the wireless connection, there is no need for a connector/cable interface between the distal portion 504 and a Patient Interface Module (PIM). As a result, the intravascular device is more easily handled and steered by users.

Referring now to FIG. 9, in the illustrated embodiment the sensor module 510 includes a sensor 512 having a diaphragm 514 and associated electrical contacts 516. Generally, the sensor 512 may take any form suitable for use within an intravascular device sized and shaped for use within vessels of a patient, including piezoresistive pressure sensors, capacitive pressure sensors, optical pressures sensors, piezoelectric pressure sensors, and/or electromagnetic pressure sensors. In some implementations, the sensor 512 includes one or more features similar to the pressure sensors described in one or more of U.S. Pat. No.7,967,762, titled “ULTRA MINIATURE PRESSURE SENSOR,” U.S. patent application Ser. No. 13/415,514, titled “MINIATURE HIGH SENSITIVITY PRESSURE SENSOR,” U.S. Pat. No. 6,167,763, titled “PRESSURE SENSOR AND GUIDE WIRE ASSEMBLY FOR BIOLOGICAL PRESSURE MEASUREMENTS,” and U.S. Pat. No. 6,461,301, titled “RESONANCE BASED PRESSURE TRANSDUCER SYSTEM,” each of which is hereby incorporated by reference in its entirety.

Electrical contacts 518 of a sensor control module 520 are electrically coupled to the electrical contacts 516 of the sensing component 512. The sensor control module 520 couples to at least one antenna that is configured to facilitate wireless communication between the sensor control module 520 and an external device. For example, an adjoining structural coil could be electrically connected to the electrical contacts 522 of the sensor control module 520 and function as an antenna for the distal portion 504 of the intravascular device 500. In some instances, the structural arrangement of the coil (e.g., diameter, length, winding pitch, winding spacing, wire cross-sectional shape, wire thickness, and/or other parameters) are selected to optimize wireless transmission for a particular wireless protocol by, for example, taking into consideration the frequency range and/or power of desired wireless transmissions. In other instances, the coil does not act as an antenna for the intravascular device. The external device is part of processing system in some instances. In some instances, the external device is an intermediary between the intravascular device 500 and the processing system. Generally, the processing system is configured to process data obtained by the sensor module 510 (including pressure sensor 512 in the illustrated embodiment) and may take the form of any suitable computer processing system, including desktop, laptop, tablet, handheld device, mobile phone, server, other hardware components, and/or combinations thereof and may be implemented utilizing local software application(s), networked software application(s), and/or cloud-based software application(s).

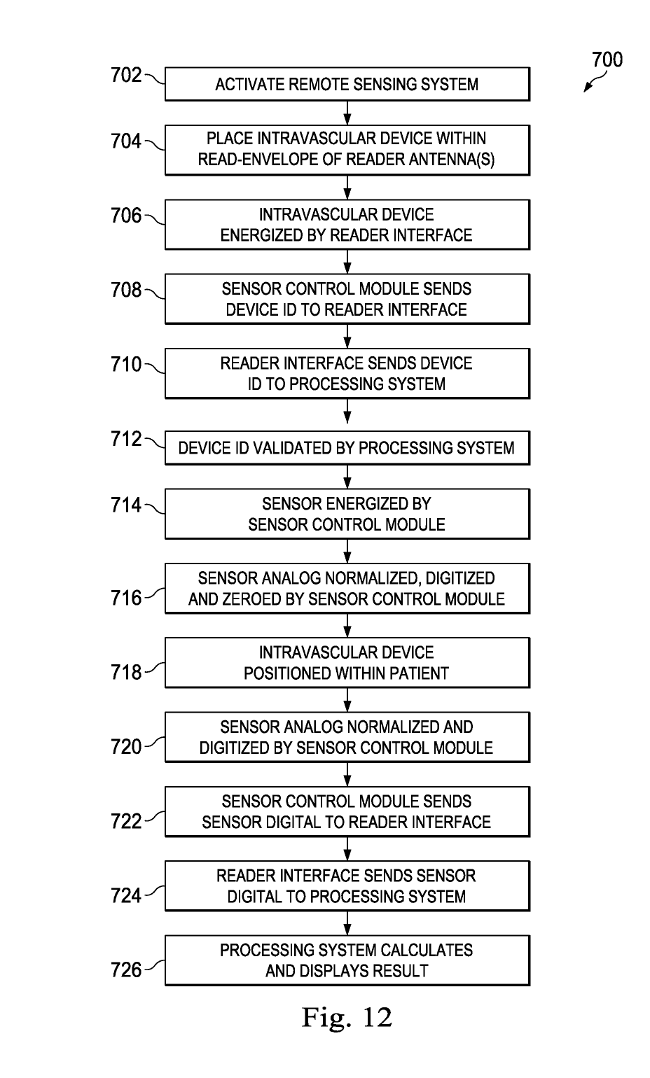

Sensor module 510 is inert in the absence of an external energy source. To that end, one or more sensor modules 510 can be energized by an external device. The sensor control module 520 can then be queried by the external device to extract sensor specific setup parameters which the processing system employs to calibrate the sensor module 512. With the pressure sensor(s) 512 activated, the sensor control module 520 is utilized to, store sensor usage data and to communicate with the external device. Additional features related to using the intravascular device 500 will be described below in the context of FIGS. 11 and 12.

Referring now to FIG. 10, shown therein is the distal portion 504 coupled to a plurality of different proximal portions. In particular, the distal portion 504 is shown being coupled to a proximal portion 550 via a connector portion 552. Similarly, the distal portion 504 is shown being coupled to a proximal portion 554 via the connector portion 552. In this regard, the connector portion 552 is representative of a standardized connection arrangement that allows the distal portion 504 to be coupled to any proximal portion having the connector portion 552. The connector portion 552 is configured to allow the distal portion 504 to be fixedly secured to the proximal portion(s). To that end, the connector portion 552 may facilitate mechanical coupling, chemical bonding, and/or otherwise securing a connection between the distal portion 504 and the proximal portion(s). In the illustrated embodiment, the connector portion 552 includes a coil structure that is threaded onto a mating coil structure of the distal portion 504. It is understood that in addition to the coil interface, the distal portion 504 may be welded and/or bonded to the connector portion 552 and/or other part of the proximal portion using an adhesive (e.g., epoxy, glue, etc.), solder, and/or other suitable bonding agent.

The standardized connection arrangement provided by connector portion 552 allows the distal portion 504 to be connected to the proximal portion of any intravascular device that includes the connector portion. Accordingly, the particular characteristics of the proximal portion can be selected based on user preference, procedure needs, and/or other parameters. Since the distal portion 504 provides full sensing and may include communication functionality without the need for communication lines extending through the proximal portion, proximal portions having different handling characteristics can be utilized. In that regard, to date guide-wires containing pressure sensors, imaging elements, and/or other electronic, optical, or electro-optical components have suffered from reduced performance characteristics compared to standard guide-wires that do not contain integrated electronics. For example, the handling performance of state-of-the-art guide-wires containing electronic components have been hampered, in some instances, by the limited space available for the core wire after accounting for the space needed for the conductive bands, spacers, conductors, sensor(s), and electronic component(s).

Further, in a similar manner, the connector portion 552 allows a particular proximal portion to be connected to any one of a plurality of different distal portions configured to mate with the connector portion 552. For example, distal portions having varying features, such as type(s) of sensing element(s), arrangement of sensing element(s), structural arrangements (e.g., outer diameter, length, mounting arrangements, flexible member type, etc.), and/or other features, may be selected based on user preference, procedure needs, and/or other factors. By providing both a plurality of available proximal portions having varying characteristics and a plurality of available distal portions having varying characteristics, a family of intravascular devices having the most desirable combination of features can be provided. This approach can be utilized to simply manufacturing processes (e.g., separating the manufacturing of the proximal and distal portions and then having an assembly stage where the various combinations of proximal and distal portions are assembled together) and/or allow a user-selectable intravascular device to be assembled in the context of a particular procedure based on a plurality of available proximal and distal portions.

Referring now to FIGS. 11 and 12, aspects of using an intravascular device, such as the intravascular devices described in the context of FIGS. 8-10, will be described in accordance with embodiments of the present disclosure. As shown in FIG. 11, at least the distal portion 504 of the intravascular device 652 is positioned within a patient 604. A boundary 601 represents the distinction between regions inside the patient 604 and regions outside the patient 602. In that regard, it is understood that the depending on the configuration of the intravascular device 652, that (1) all of the electrical, optical, and/or electro-optical elements are positioned inside the patient 604 during a procedure or (2) some of the electrical, optical, and/or electro-optical elements are positioned inside the patient 604 while at least a portion of one or more of the electrical, optical, and/or electro-optical elements are positioned in the region 602 outside the patient during a procedure. For example, in the illustrated embodiment the antenna 612 is partially positioned inside the patient 604 and partially positioned outside the patient in region 602 as the antenna extends through section 603 of the patient boundary 601. In other implementations, the antenna 612 and/or the sensor control module 610 are positioned entirely in the region 602 outside the patient 604. It is understood that the particular region inside the patient 604 will depend on the size and type of intravascular device 652 being utilized, but in general the region inside the patient 604 can be any vasculature, cavity, passageway, or other area of interest. Accordingly, it is also understood that the exact distance from the distal portion 504 of the intravascular device 652 to the region outside the patient 602 will vary. Further, the air-interface protocol between the intravascular device(s) 652 and the reader interface 654 and between the reader interface 654 and the processing system 656 may include any one or combination of the following: ISO18000-2 (130 kHz), IS018000-3 (13.56 MHz), IS018000-4 (2.4 GHz), ISO18000-6c (870-930 MHz).

In the illustrated embodiment of FIG. 11, the guide-wire antenna 612 is configured to harvest energy from the reader antenna 616, and communicate with the reader antenna 616. The reader antenna 616 is strategically oriented and positioned near the guide-wire antenna 612 to optimize the energy and data transmission between the processing system and an in-use intravascular device 652. The reader antenna 616 may be rigid or flexible, reusable or disposable, and may be deployed as a plurality of antennas, antenna types, and antenna locations. The reader antenna 616 may be within the following dimensions: 150-500 mm wide, 150-2000 mm long, and 1-100 mm thick. The reader antenna 616 may be mounted beneath the mat that separates the patient from the operating table, mounted onto the underside of the operating table, integrated into the mat, integrated into the top-side or bottom-side of the operating table, mounted on the floor, mounted on the ceiling or in the crawl-space above the ceiling, mounted inside or to an outer surface of the operating table pedestal, mounted to the monitor boom on a side or behind the monitors, mounted to an articulating ceiling or bedside arm, mounted to a bedside pole a roll-around IV pole or processing system 656. The reader antenna 616 communicates with a reader module 618 via a cable interface 620 or other suitable connection. The reader module 618 receives power from a power source 658, which may be an AC outlet, power-over-Ethernet (POE), or suitable power supply.

Energy harvested by guide-wire antenna 612 energizes sensor control module 610 and sensing component 608. In some instances, a telemetry module 660 communicates sensor output as received by reader module 618 to a processing system 656 via an antenna 662. In particular, a system antenna 664 of the processing system 656 that is in communication with a processing component 666 communicates with the antenna 662. In some instances, the processing system 656 is a hemostat system, such as Siemens AXIOM Sensis, Mennen Horizon XVu, and Philips Xper IM Physiomonitoring 5. In some particular instances, the processing system 656 is configured to obtain pressure data related to a vessel of the patient in which the distal portion 504 is positioned. In some particular instances, the processing system 656 utilizes data from the hemostat system and the intravascular device 652 to calculate fractional flow reserve (FFR).

Referring now to FIG. 12, a method 700 of performing a medical procedure in accordance with the present disclosure will be described. It is understood that the “reader antenna,” “intravascular device,” and associated elements (e.g., “sensor control module”, “device ID”, “sensor,” etc.) referred to may be singular or plural in some embodiments. In that regard, the method described below encompasses the use of some of the exemplary embodiments of intravascular devices described in the present disclosure. Accordingly, a more specific understanding of the steps of the method may be achieved by considering the particular features of one or more of the exemplary intravascular devices described above. It is also understood that the steps of method 700 are exemplary in nature and that one or more of the steps may be omitted, one or more additional steps may be added, and/or the order of the steps may be changed without departing from the scope of the present disclosure.

At step 702, the remote sensing system is activated. At step 704, the intravascular device is placed within the read-envelope of the reader antenna. At step 706, the intravascular device is energized by the reader interface. At step 708, the sensor control module sends the device ID for the intravascular device to the reader interface. At step 710, the reader interface sends the device ID to the processing system. At step 712, the device ID is validated (or not) by processing system. If the device ID is not validated, then the procedure is stopped or more information may be required for the procedure to continue. At step 714, the sensor of the intravascular device is energized by the sensor control module of the intravascular device. At step 716, the sensor analog data is normalized, digitized, and zeroed by the sensor control module. At step 718, at least the distal portion of the intravascular device containing the sensor is positioned within the patient. At step 720, the sensor analog data obtained within the patient is normalized and digitized by the sensor control module. At step 722, the sensor control module sends the digital data corresponding to the analog sensor data to reader interface. In some instances, the sending of the digital data is performed in real time. In other instances, the digital data is stored, either temporarily or permanently, locally within the intravascular device and later retrieved by the reader interface. At step 724, the reader interface sends the digital sensor data to the processing system. At step 726, the processing system calculates and displays the sensor data.

In some instances a unique identifier and usage history are stored in the memory of the intravascular device. When the intravascular device is energized, all sensing and/or imaging capabilities are disabled by default, and enabled or activated only after the unique identifier and relevant usage parameters are vetted and approved by the processing system. The vetting process helps prevent the use of counterfeit goods, and/or use of non-sterilized goods, and/or use of expired goods, and/or overuse of goods, etc. The stored elements might include, but are not limited to the following manufacturing assigned elements: device ID, usage limit, sensor ID, temperature coefficient, zero offset, scale factor, sensitivity, manufacture date, manufacture time, and manufacture location. In addition, the stored elements might include, but are not limited to the following time-of-use data elements: use count, date, time, location, system ID, pressure minimum, pressure maximum, velocity minimum, velocity maximum, temperature minimum, temperature maximum, centered (y/n), and reader ID.

Energized sensing element(s) detect local environmental parameters. In some instances, the at least one sensing element of the intravascular device is moved through the region of interest (e.g., a pullback is performed) during data acquisition. In some instances, movement of the proximal portion of the intravascular device is monitored, or movement of the guide-wire antenna is measured by reader antenna(s). The measured movement is correlated to the relative position of the associated sensor through the region of interest. In that regard, since the relative position of the guide-wire antenna may be fixed relative to the at least one sensing element, the movement of the guide-wire antenna can be utilized as proxy for movement of the at least one sensing element.

Persons skilled in the art will also recognize that the apparatus, systems, and methods described above can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure.

What is claimed is:

1. A pressure-sensing guide-wire, comprising:
an elongate flexible element having a proximal portion and a distal portion, the elongate flexible element having an outer diameter of 0.018″ or less;
a pressure sensing component coupled to the distal portion of the elongate flexible element;
a sensor control module coupled to the elongate flexible element, the sensor control module being in electrical communication with the pressure sensing component and storing information about the pressure sensing component; and
at least one conductor having a proximal section and a distal section, wherein the distal section of the at least one conductor is coupled to the sensor control module and the proximal section of the at least one conductor is coupled to at least one connector.

2. The guide-wire of claim 1, wherein the sensor control module includes an electrically erasable programmable read-only memory (EEPROM).

3. The guide-wire of claim 2, wherein the information about the pressure sensing component includes calibration information.

4. The guide-wire of claim 3, wherein the at least one conductor consists of three to five conductors.

5. The guide-wire of claim 4, wherein the at least one connector consists of three to five connectors.

6. The guide-wire of claim 5, wherein the three to five connectors each comprise a conductive band.

7. An intravascular pressure-sensing system, comprising:
a pressure-sensing guide-wire including:
an elongate flexible element having a proximal portion and a distal portion, the elongate flexible element having an outer diameter of 0.018″ or less;
a pressure sensing component coupled to the distal portion of the elongate flexible element;
a sensor control module coupled to the distal portion of the elongate flexible element, the sensor control module being in electrical communication with the pressure sensing component and storing information about the pressure sensing component; and
at least one conductor having a proximal section and a distal section, wherein the distal section of the at least one conductor is coupled to the sensor control module and the proximal section of the at least one conductor is coupled to at least one connector;
a processing system configured to receive the information originating at the sensor; and
an interface configured to communicatively couple the sensor to the processing system such that the sensor output is conditioned and communicated to the processing system.

8. The system of claim 7, wherein the sensor control module includes an electrically erasable programmable read-only memory (EEPROM).

9. The system of claim 8, wherein sensor specific coefficients for temperature, and/or zero offset, and/or scale factor, and/or sensitivity are stored in memory

10. The system of claim 9, wherein the at least one conductor includes three to five conductors.

11. The system of claim 10, wherein the at least one connector includes three to five connectors.

12. The system of claim 11, wherein the three to five connectors each comprise a conductive band.

13. The system of claim 7, wherein the sensor control module normalizes the sensor output using at least one of a temperature coefficient, a zero offset coefficient, a scale factor coefficient, and a sensitivity coefficient stored in the memory of the sensor control module.

14. The system of claim 7, wherein the interface comprises a communication cable.

15. The system of claim 14, wherein the communication cable includes a first connector portion for interfacing with the at least one connector of the pressure-sensing guide-wire and a second connector portion for interfacing with a component of the processing system.

16. The system of claim 15, wherein the component of the processing system is a patient interface module (PIM).

17. A method, comprising:
obtaining information about a pressure sensing component of a pressure-sensing guide-wire having an outer diameter of 0.018″ or less from a sensor control module coupled to a pressure-sensing guide-wire, the pressure sensing component being coupled to the distal portion of the pressure-sensing guide-wire; and
normalizing data received from the pressure sensing component based on the information about the pressure sensing component stored in the memory of the sensor control module.

18. The method of claim 17, wherein the information about the pressure sensing component includes calibration information about the pressure sensing component.

19. The method of claim 18, wherein the sensor control module includes an electrically erasable programmable read-only memory (EEPROM).
